(19) United States
(12) Patent Application Publication
(10) Pub. No.: US 2010/0010320 A1
(43) Pub. Date: Jan. 14, 2010

(54) MOBILE MEDICAL WORKSTATION AND A TEMPORARILY ASSOCIATING MOBILE COMPUTING DEVICE

(76) Inventors: David G. Perkins, Tully, NY (US); Saverio J. Carello, Syracuse, NY (US)

Correspondence Address: HISCOCK & BARCLAY, LLP, ONE PARK PLACE, 300 S. STATE ST., SYRACUSE, NY, 13202, US

(21) Appl. No.: 12/498,851

(22) Filed: Jul. 7, 2009

Related U.S. Application Data
(60) Provisional application No. 61/078,976, filed on Jul. 8, 2008.
(60) Provisional application No. 61/134,229, filed on Jul. 7, 2008.

(51) Int. Cl. A61B 5/00

(52) U.S. Cl. 600/300

(57) ABSTRACT

A system, method and software instructions that enable a mobile computing device to communicate with and search a vital signs measuring device for any physiological data that has been obtained from one or more patients and that has not yet been communicated to another device, such as to a electronic medical record repository. Further, the data is accessed by and stored into the mobile computing device for review, logging and selective transmission to other devices including such as the electronic medical record repository.

### CROSS-REFERENCE TO RELATED APPLICATIONS

This patent application claims priority to U.S. Provisional Patent Application having Ser. No. 61/134,229 and identified by attorney docket matter number 3030290 US01, and that is entitled “Temporary Associating Mobile Computing Device” and that was filed on Jul. 7, 2008. This patent application also claims priority to U.S. Provisional Patent Application having Ser. No. 61/078,976 and identified by attorney docket matter number 3030293 US01, and that is entitled “Temporary Associating Mobile Computing Device” and that was filed on Jul. 8, 2008. All of the aforementioned patent(s), patent application(s) and patent publication(s) are herein incorporated by reference in their entirety.

This document is a non-provisional utility patent application filed pursuant to 35 USC 111(a).

### CROSS-REFERENCE TO APPLICATIONS INCLUDING RELATED SUBJECT MATTER

This patent application includes subject matter related to that of U.S. patent application Ser. No. 11/703,470 entitled “Information Workflow for a Medical Diagnostic Workstation”, which was published as U.S. Patent Publication 2007/0185390 and is a continuation-in-part application under 37 C.F.R. 1.53(b) and claims benefit of priority under 35 U.S.C. 120 to co-pending and commonly owned U.S. non-provisional U.S. patent application Ser. No. 11/131,015 entitled “Mobile Medical Workstation”, which was published as U.S. Patent Publication No. 2005/0288571, and which claims priority under 35 U.S.C. 120 to co-pending and commonly owned U.S. non-provisional U.S. patent application Ser. No. 10/643,487 entitled “Diagnostic Instrument Workstation”, which was published as U.S. Patent Publication No. 2004/0186357. All of the aforementioned patent(s), patent application(s) and patent publication(s) are herein incorporated by reference in their entirety.

### FIELD OF THE INVENTION

This invention relates to the field of diagnostic medicine and particularly to a mobile medical diagnostic workstation and an intermittently attachable mobile computing device that are collectively employed for measuring and storing a number of physiologic parameters for at least one patient

### BACKGROUND OF THE INVENTION

In the context of a nursing shortage, patient vital signs data readings are often taken numerous times per day (as many as six or more readings) by nursing aides (also referred to as Patient Care Technicians (PCTs)) who often have no or little clinical training. Many hospitals utilize more temporary contract or “traveler” nurses who float between sites. As a result, the PCT users of the monitoring equipment are typically more numerous and transitory as compared to circumstances where the monitoring equipment is used by full time nurses. Further, PCT users are typically required to learn internal procedures of each particular health care facility, which consumes time that could otherwise be allocated to patient care.

### SUMMARY OF THE INVENTION

The invention provides a system for intermittently obtaining and communicating physiological data from a plurality of patients within a health care facility. A mobile workstation, including a docking station, is associated with each of a plurality of patients and is configured to intermittently communicate with and optionally attach to a mobile computing device carried by a health care practitioner. The foregoing as well as other objects, aspects, features, and advantages of the invention will become more apparent from the following description and from the claims.

### BRIEF DESCRIPTION OF THE DRAWINGS

The objects and features of the invention can be better understood with reference to the claims and drawings described below. The drawings are not necessarily to scale; the emphasis is instead generally being placed upon illustrating the principles of the invention. Within the drawings, like reference numbers are used to indicate like parts throughout the various views. Differences between like parts may cause those like parts to be each indicated by different reference numbers. Unlike parts are indicated by different reference numbers.

FIG. 1 is a front perspective view of the mobile medical workstation including a docking station that is unattached to mobile computing device.

FIG. 2 is a front perspective view of the mobile medical workstation of FIG. 1 where the docking station that is attached to a mobile computing device.

FIG. 3 is a rear perspective view of the mobile medical workstation of FIG. 1 showing a vital signs device measuring device and a bar code scanner included within the workstation.

FIG. 4 is a top perspective view of a visitation between a health care practitioner and a patient.

FIG. 5 illustrates a relationship between patients, workstations, mobile computing devices and health care practitioners.

### DETAILED DESCRIPTION OF THE INVENTION

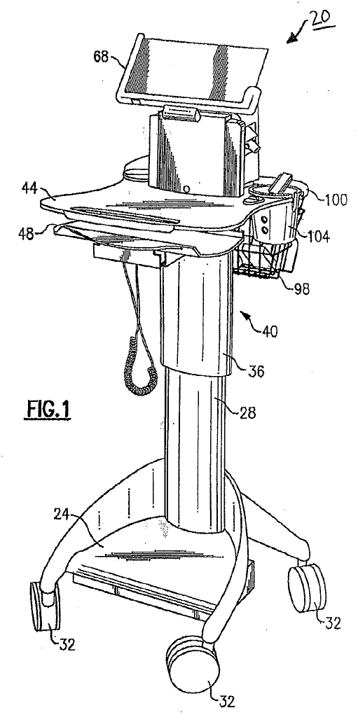

FIG. 1 is a front perspective view of the mobile medical workstation 20 including a docking station 68 that is unattached to mobile computing device. The docking station is configured to intermittently attach to one mobile computing device at any one instant in time. In this embodiment, the docking station 68 is configured to attach to a tablet mobile computing device, also referred to as a tablet personal computer or tablet (PC). In this circumstance, the docking station is said to be vacant.

As shown, the workstation 20 includes a horizontal work surface 44, a keyboard tray 48, a handle 100 for grasping and moving the workstation 20, a basket 98, and a storage container 104. The workstation 20 has a supporting structure 28 that includes a lower base 24, a post member 36 and an upper portion 40 and a set of wheels (casters) 32. The workstation 20 is configured to receive electrical power (120 AC) from a wall outlet or to operate from a battery (not shown).

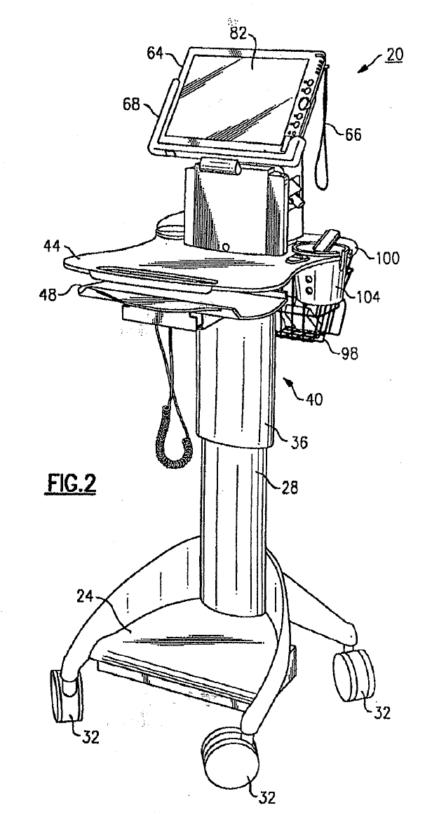

FIG. 2 is a front perspective view of the mobile medical workstation of FIG. 1 where the docking station is attached to a tablet mobile computing device (PC) 64. As shown, the tablet PC 64 includes an integrated user interface display 82 and a tethered writing stylus 66. The workstation 20 also includes a vital signs measuring device (not shown).

The workstation 20 is configured to intermittently receive within its docking station 68, any selected one of a plurality of (mobile computing devices) tablet PCs 64, at any one time. As a result, the workstation 20 is configured to receive engage, interoperate and communicate with each of a plurality of different mobile computing devices (tablet PCs) 64 over time.

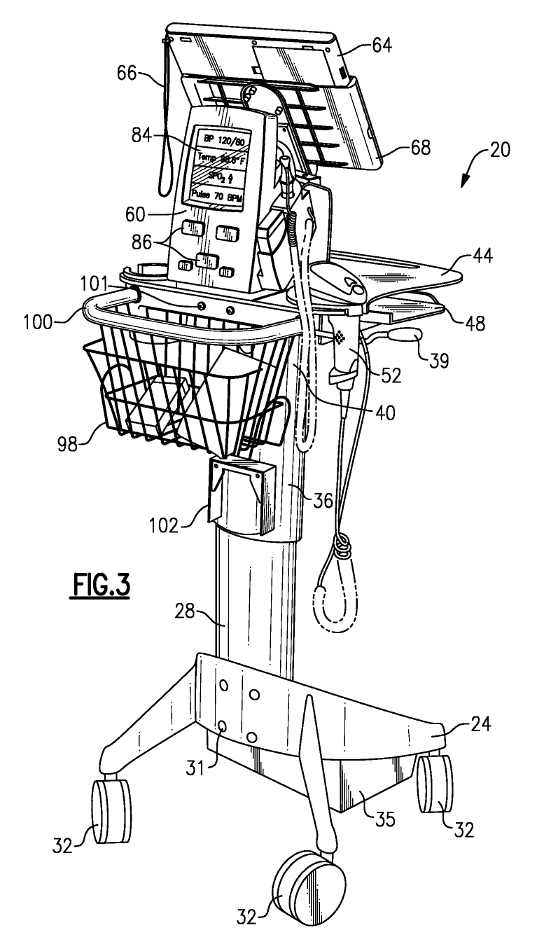

FIG. 3 is a rear perspective view of the mobile medical workstation of FIG. 1 showing a vital signs monitoring device 60 and a bar code scanner 52 included within the workstation. The vital signs measuring device 60 measures a patient's vital signs including blood pressure, temperature, pulse oximetry. In one embodiment, the vital signs measuring device is SPOT LXI vital signs measuring device supplied by Welch Allyn of Skaneateles, N.Y.

The tablet PC 64 and the vital signs measuring device 60 are interconnected via at least one wireline communications channel (not shown). The communications channel is implemented as at least one of a serial and/or a universal serial bus (USB) communications path. In some embodiments, other types of wireline connections are employed to implement the communications channel.

In some embodiments, the vital signs measuring device 60 includes at least one wireless communications channel (not shown). The wireless communications channel is implemented as at least one of a Bluetooth, Zigbee, WiFi, or an RFID communications path. The vital signs measuring device 60 can include a separate microcontroller therein and/or can directly receive input commands via software contained with the tablet PC 64.

In some embodiments, a user logs into the mobile computing device (tablet PC) 64 in order to access its functionality. In some embodiments, the tablet PC 64 is configured to accept or reject a logon request of the user, and has within its memory a list of users (health care practitioners) that are permitted to log onto and use that particular PC 64. This list is also referred to as a “pick list”.

In some embodiments, the PC 64 communicates with another computing entity, such as a network server, to determine if the user is permitted to log onto that particular device 64. Optionally, the network server downloads to the tablet PC 64, a list of users and associated information, such as password information, that are permitted to use the tablet PC 64. Communication between the tablet PC and the network is performed via a wireline or a wireless communication channel.

In these embodiments, a downloaded “pick list” is periodically updated at the network server. This arrangement enables a network server to pre-assign users to particular mobile devices 64. For example, nurses that are located on a particular floor within a health care facility can be listed into a pick list that is downloaded into to the mobile devices 64 associated with that particular floor. The pick list can be pre-loaded onto the mobile devices 64 before a working shift is to begin.

In some embodiments, the tablet PC 64 includes a scanning device, such as a bar code scanner (not shown). This bar code scanning device is not to be confused with the bar code scanner 52 that is attached to the workstation 20 and shown in FIG. 3. In one use embodiment, the user logs onto the tablet PC 64 via the bar code scanner (not shown) attached to the tablet PC. In this embodiment, a bar code symbol that identifies the user (health care practitioner) is scanned via the bar code scanner (not shown) of the tablet (PC) 64. The bar code symbol includes sufficient information to perform a log on operation. Optionally, the bar code symbol is located onto an identification card worn or possessed by the user.

Upon logon to the mobile device 64, the bar code scanner of the tablet PC can be further employed to scan and identify other entities, such as a patient and/or a workstation 20. Upon visiting a patient, the user employs the bar code scanner of the tablet PC 64 to scan a patient bar code symbol. The patient bar code symbol identifies the patient and provides sufficient information to associate patient related information within an electronic medical record (EMR). The patient bar code symbol is attached to, disposed upon, proximate to and/or associated with the patient in some manner. Optionally, the patient bar code is located on an identification card worn or possessed by the patient.

Also, upon visiting a patient, the user employs the bar code scanner of the tablet PC 64 to scan a workstation bar code symbol. The workstation bar code identifies the workstation and provides sufficient information to associate workstation related information, including information associated with the vital signs monitor 60 within the workstation 20, within the information content of an electronic medical record (EMR). The workstation bar code symbol is attached to, disposed upon or associated with the work station 20.

In some embodiments, the tablet PC 64 itself, further includes an RFID reader (not shown). The RFID reader is used in the same manner as described for the bar code scanner of the tablet PC 64. The PC 64 includes an internal clock which is periodically synchronized with an external clock. In this embodiment,

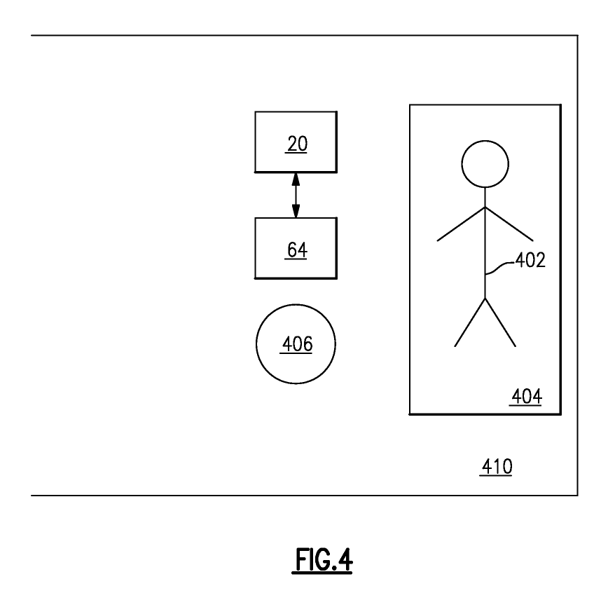

FIG. 4 is a top perspective view of a visitation between a health care practitioner 406 and a patient 402. In this use scenario, the health care practitioner 406, also referred to as a user 406, is an operator (user) of the tablet PC 64. As shown, the patient 402 is lying upon a bed 404 within a health care facility 410.

Typically, before the visit with this patient, the user 406 has previously logged onto the tablet PC 64. During the visit with a patient 402, the user 406 optionally engages (docks) the tablet PC 64 to the docking station 68 of the workstation 20. While engaged, the tablet PC 64 can receive an electrical power charge from the docking station 68.

In some embodiments, the docking station has a locking mechanism to physically secure the tablet PC 64. This can be useful, for example, when the user 406 leaves the area 410 proximate to the patient and elects to leave the tablet PC 64 engaged (docket) into the docking station 68.

While the tablet PC 64 is engaged (docked) into the docking station 68. In some embodiments, the user 406 enters a command into the PC 64 to communicate a set of information, including readings of physiological data that were obtained from the patient 402 during a prior period of time by the vital signs device 60 of the workstation 20. Optionally, the user 406 selects particular physiological readings to further communicate (import) to a repository of patient records, such as to an electronic medical records (EMR) server. In some circumstances, the server is implemented as a node within a wireline or wireless network.

Physiological readings that are not further communicated are by default, are stored into a log file of the tablet PC 64. Physiological readings can be stored into the log file automatically or on demand. As stored within the PC 64 and/or further communicated to a remote server, the set of information received from the vital signs 60 device is associated with the identity of the patient 402, the workstation 20 and the user 406. Each set of information is stored into non-volatile memory within the tablet PC 64.

The set of information received during a visitation of a patient includes one or more readings of physiological data, representing one or more readings of measured physiological parameters that were obtained at one or more points in time prior or during the visitation, from the patient 402 via the vital signs monitor 60. The set of information received by the tablet PC 64 is typically communicated from the vital signs device 60 to the PC 64 via a wireline communications channel, such as via a serial (RS-232) channel or universal serial bus (USB) channel, or alternatively can be communicated via a wireless communications channel such as WiFi, BlueTooth or Zigbee, for example.

In one embodiment, the tablet PC 64 includes a wireless radio and communicates wirelessly to other nodes within a wireless network. In another embodiment, the tablet PC is attached to a wireline connection and communicates over the wireline connection to one or more nodes within a network.

In some use scenarios, the user 406 visits each of a plurality of patients 402, typically one patient at a time, over a period of time, within a working shift of the user 406. As described above, a set of information associated with each patient that is visited, is communicated from a workstation 20 associated with the patient that is visited, to the tablet PC 64 associated with the user 406, during the user's visit with each patient 402. After the user 406 has visited each of the plurality of patients, the tablet PC 64 has received a set of information from each of a plurality of workstations 20 that are each respectively associated with each patient visitation. Collectively, the a union of multiple sets of information is referred to as aggregate information.

The aggregate information received by the tablet PC 64, representing information that is associated with a plurality of patient visitations, is later communicated from the tablet PC 64 to another entity (computer), such as a network server. In some embodiments, the PC 64 is configured to communicate information via a communications path that is separate from that provided by a docking station 68. For example, the PC 64 can communicate via a wireline or wireless communications port included within the tablet PC 64, that is separate from that provided by the docking station 68. As a result, when information is communicated from the tablet PC 64, the tablet PC 64 may or may not be docked into any docking station 68, and if docked into a docking station 68, the tablet PC 64 is not necessarily docked into a docking station 68 of any workstation 20 or not docked into a docking station 68 of a workstation 20 that is a source of data that is currently being communicated to another entity.

In some embodiments, software within the tablet PC 64 searches for and identifies information that has been received and has been stored for a configurable period of time and that has not yet been communicated to another entity, such as a network server. In some embodiments, the period of time is configured to be equal to 60 minutes.

In some embodiments, in accordance with a configuration parameter, when the software identifies such information, it automatically attempts to transmit the information to a another entity, such as a network server, if a communications channel to that other entity is available. For example, if the PC 64 has a working wireless communications port, then the software initiates communication with a network server, providing that the network server is available via a wireless communications channel.

When communicating information, the associated patient, workstation and user identifiers are also communicated with the received information. Optionally, the user and workstation identifiers (numbers) are mapped to character string representations (names) of the user, workstation and patient and are also communicated.

If a wireless communications port is unavailable or not functioning, or if the network server is unavailable, then the software generates a reminder to the user that the information is due to be transmitted another entity. In some use scenarios, in response to such a reminder, the user would initiate communication with a network server through a wireline connection.

Optionally and in accordance with a configuration parameter, the software periodically attempts to establish a wireless connection to the network server. Or, to save battery power and in accordance with a configuration parameter, the tablet PC 64 notifies the user when a communications channel is available and initiate communication upon approval of the user.

In other embodiments, in accordance with a configuration parameter, when the software identifies such information, it automatically generates a reminder to the user that the information received is due to be transmitted to the network server. The reminder can be in the form of a popup window and/or an audible sound to notify the user of the PC 64. In some embodiments, during a period of time while communicating with the network server, an internal clock within the tablet PC 64 is synchronized with a clock of a network server,

In some embodiments, the mobile computing device is implemented as a tablet PC supplied by Motion Computing of Austin, Tex. In one embodiment, the table PC 64 is a Motion C5 computer.

In some embodiments, the vital signs measuring device 60 communicates with the tablet PC 64 via a wireless communication channel. The wireless communications channel is implemented via WiFi, BlueTooth or Zigbee, for example, or via other wireless methods. In some embodiments, the tablet PC 64 initiates device discovery of the vital signs measuring device 60, upon coming into physical proximity to vital signs measuring device 60 that is located within a workstation 20 associated and proximate to a patient. In other embodiments, device discovery of a tablet PC 64 is initiated by the vital signs measuring device 60 when in physical proximity to a tablet PC 64.

Upon completion of device discovery, a device association protocol is exercised between the vital signs measuring device 60 and the newly proximate PC 64. Upon completion of the device association, the PC 64 queries the vital signs measuring device 60 for information obtained regarding the patient 402. Upon termination of the patient visit, a device dis-association protocol is exercised between the PC 64 and the vital signs measuring device 60.

In other embodiments, a device discovery protocol is initiated via a barcode or RFID reading performed by the PC 64. In one embodiment, the barcode encodes information that identifies the vital signs measuring device 60 and provides information to establish a wireless communications session with the device 60. In another embodiment, the RFID tag encodes information that identifies the vital signs measuring device 60 and provides information to establish a wireless communications session with the device 60. In some embodiments, the barcode and/or/ RFID tag are attached or disposed proximate to the vital signs measuring device 60.

In some embodiments, the information that provides information to establish a wireless communications session includes the identification of one or more wireless communications protocol(s) and of one or more address(s) within those protocol(s) that are supported by the vital signs measuring device 60. This type of information enables the PC 64 to quickly and unambiguously establish communication with each vital signs measuring device 60 it comes within proximity to.

In one use embodiment, a user carrying a tablet PC 64 walks up to a workstation 20 including a vital signs measuring device 60, enters a command into the PC 64 to establish device association between the PC 64 and the vital signs measuring device 60 and employs the barcode reader of the tablet PC 64 to a scan a barcode symbol attached to the vital signs measuring device 60 to obtain information as input into the command to establish device association. The same type of procedure can be employed where instead of a bar code being scanned, an RFID tag attached to the vital signs measuring device 60 is read to obtain information that is input into the command to establish device association.

In another use embodiment, bar code symbol that identifies the user (health care practitioner) is scanned via the bar code scanner 52 of the mobile device (PC) 64. Optionally, the bar code symbol is located onto an identification card worn or possessed by the user.

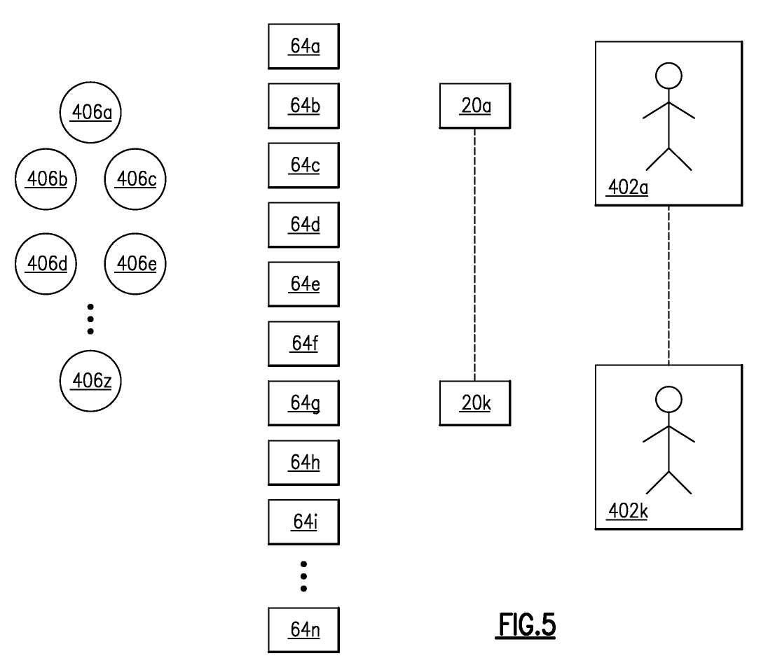

FIG. 5 illustrates a relationship between patients 402a-402k, workstations 20a-20k, mobile computing devices (tablet PCs) 64a-64n and health care practitioners (users) 406a-406z. In some use scenarios, a first plurality of mobile computing devices (tablet PCs) 64a-64n are used and shared by a second plurality of health care practitioners (users) 406a-406z. For example, (6) tablet PCs 64a-64f can be shared among (18) health care practitioners 64a-64r.

Optionally, each tablet PC 64 64a-64n can be assigned to a separate subgroup of (3) health care practitioners 406a-406z. As a further option, each member of the subgroup can be assigned different working hours, such as the daytime, evening or midnight working shift, for example. Or alternatively, each tablet PC 64a-64n is not assigned to one or more health care practitioners 406a-406z and can be used on demand by a health care practitioner 406a-406z, if available.

Note that various types of mobile computing devices can be employed, such as hand held personal digital assistants, laptop computers etc. providing that such a device is capable of communicating with a vital signs device as described above.

The following summarizes some of the aspects of the invention. In some embodiments, the invention provides for a system and a method for managing physiological data obtained from each of one or more patients, that includes at least one vital signs measuring device that is configured to obtain and store physiological data associated with one or more patients, a mobile computing device that is configured to communicate with the vital signs measuring device, and a software program executing within the mobile computing device.

The software program can be stored onto a computer readable storage medium and is configured to communicate commands to the vital signs measuring device and configured to search the vital signs measuring device for the physiological data that has been obtained and that has not yet been communicated to another device by the vital signs monitoring device and further configured to access and store the data within the mobile computing device. Another device can be an electronic medical records server.

In some embodiments, the vital signs measuring device and the mobile computing device communicate via a wireless communications channel. In other embodiments, the system includes a docking station that is electronically attached to the vital signs measuring device and that interfaces with the mobile computing device. The vital signs measuring device and the mobile computing device communicate via the docking station.

Optionally, the mobile computing device transmits data selected by a user of the mobile computing device to the another device upon demand of the user and/or the mobile computing device logs data that is selected by a user of the mobile computing device.

In some use scenarios, the mobile computing device periodically attempts to re-transmit the data in response to a transmission error associated with the another device and/or the mobile computing device periodically reminds the user of data that has not yet been transmitted from the mobile computing device to another device.

While the present invention has been explained with reference to the structure disclosed herein, it is not confined to the details set forth and this invention is intended to cover any modifications and changes as may come within the scope and spirit of the following claims.

What is claimed is:

1. A system for managing physiological data obtained from each of one or more patients, comprising:
at least one vital signs measuring device that is configured to obtain and store physiological data associated with one or more patients;
a mobile computing device that is configured to communicate with said vital signs measuring device; and
a software program executing within said mobile computing device, said program is configured to communicate commands to said vital signs measuring device and configured to search said vital signs measuring device for said physiological data that has been obtained and that has not yet been communicated to another device by said vital signs monitoring device; and where said program is further configured to access and store said data within said mobile computing device.

2. The system of claim 1 wherein said vital signs measuring device and said mobile computing device communicate via a wireless communications channel.

3. The system of claim 1 further including a docking station that is electronically attached to said vital signs measuring device and that interfaces with said mobile computing device and wherein said vital signs measuring device and said mobile computing device communicate via said docking station.

4. The system of claim 1 wherein said mobile computing device transmits data selected by a user of said mobile computing device to said another device upon demand of said user.

5. The system of claim 1 wherein said mobile computing device logs data that is selected by a user of the mobile computing device;

6. The system of claim 1 wherein said mobile computing device periodically attempts to re-transmit said data in response to a transmission error associated with said another device.

7. The system of claim 6 wherein said mobile computing device periodically reminds said user of data that has not yet been transmitted from said mobile computing device.

8. The system of claim 1 wherein said another device is an electronic medical records server.

9. A method for managing physiological data obtained from each of one or more patients, the method comprising:
communicating commands to a vital signs measuring device;
searching said vital signs measuring device for said physiological data that has been obtained and that has not yet been communicated to another device by said vital signs measuring device;
accessing said data within a mobile computing device that is configured to communicate with said vital signs measuring device; and
storing said data within said mobile computing device.

10. The method of claim 1, wherein said vital signs measuring device and said mobile computing device communicate via a wireless communications channel.

11. The method of claim 1, wherein a docking station is electronically attached to said vital signs measuring device and interfaces with said mobile computing device; and wherein said vital signs measuring device and said mobile computing device communicate via said docking station.

12. The method of claim 1, further comprising transmitting data selected by a user of said mobile computing device to said another device upon demand of said user.

13. The method of claim 1, further comprising logging data that is selected by a user of the mobile computing device.

14. The method of claim 1, further comprising periodically attempting to re-transmit said data in response to a transmission error associated with said another device.

15. A computer-readable storage medium containing computer executable instructions which when executed by a computer perform a method of managing physiological data obtained from each of one or more patients, the method comprising the steps of:
communicating commands to a vital signs measuring device;
searching said vital signs measuring device for any physiological data that has been obtained and that has not yet been communicated to another device by said vital signs measuring device;
accessing said data from within a mobile computing device that is configured to communicate with said vital signs measuring device; and
storing said data within said mobile computing device.

16. The computer-readable storage medium of claim 15, wherein said vital signs measuring device and said mobile computing device communicate via a wireless communications channel.

17. The computer-readable storage medium of claim 15, wherein a docking station is electronically attached to said vital signs measuring device and interfaces with said mobile computing device; and wherein said vital signs measuring device and said mobile computing device communicate via said docking station.

18. The computer-readable storage medium of claim 15, further comprising transmitting data selected by a user of said mobile computing device to said another device upon demand of said user.

19. The computer-readable storage medium of claim 15, further comprising logging data that is selected by a user of the mobile computing device.

20. The computer-readable storage medium of claim 15, further comprising periodically attempting to re-transmit said data in response to a transmission error associated with said another device.
